(19) United States
(12) Patent Application Publication
(10) Pub. No.: US 2007/0172436 A1
(43) Pub. Date: Jul. 26, 2007

(54) Nonaqueous ascorbic acid compositions and methods for preparing same

(76) Inventor: Jerry Zhang, Grayslake, IL (US)

Correspondence Address: Jerry Zhang, 1061 Chadwick Dr., Grayslake, IL, 60030, US

(21) Appl. No.: 11/337,786

(22) Filed: Jan. 23, 2006

(51) Int. Cl. A61K 31/375; A61K 8/49; A61K 31/385; A61K 31/355; A61K 31/07; A61K 38/43

(52) U.S. Cl. 424/59; 514/474; 424/94.1; 514/725; 514/440; 514/458

(57) ABSTRACT

A method for preparing a nonaqueous ascorbic acid composition in an alcohol solvent comprising ascorbic acid, a solubilization enhancer, and an oleaginous skin protectant is described. The amount of ascorbic acid dissolved in the nonaqueous composition in the presence of the solubilization enhancer is greater than the amount of ascorbic acid that would be soluble in the solvent in the absence of the solubilization enhancer. A nonaqueous composition comprises the dissolved ascorbic acid, solubilization enhancer, and homogenized oleaginous skin protectant.

### CROSS-REFERENCE TO RELATED APPLICATIONS

### Not Applicable

### FEDERALLY SPONSORED RESEARCH

Not Applicable

### SEQUENCE LISTING OR PROGRAM

### Not Applicable

### FIELD OF INVENTION

This invention concerns a method for preparing a nonaqueous ascorbic acid composition in an alcohol solvent comprising a solubilization enhancer and an oleaginous skin protectant. A nonaqueous topical composition comprises the dissolved ascorbic acid, solubilization enhancer, and homogenized oleaginous skin protectant in the alcohol solvent.

### BACKGROUND OF THE INVENTION

Ascorbic acid, Vitamin C, is known as being suitable for preventing or treating photo-damage to skin. Ascorbic acid is highly soluble, but extremely unstable, in water and rapidly degrades to bio-inactive products. It is known that ascorbic acid is much more stable in nonaqueous organic solvents. Unfortunately, its solubility is limited in such solvents, generally no more than a few percent by weight. Since the beneficial effects of ascorbic acid are known to be dose-dependent, it is therefore highly desirable to prepare nonaqueous topical compositions comprising ascorbic acid dissolved in dermatologically acceptable carriers at a concentration of greater than 10% by weight, more preferably up to 20 to 40% by weight.

Numerous patents and publications disclose various ways of stabilizing ascorbic acid, especially for low concentrations of ascorbic acid. Because of the solubility limitation, a number of studies disclose using suspension of particulate ascorbic acid in nonaqueous organic solvents as ways to prepare topical compositions of higher ascorbic acid contents.

For example, U.S. Pat. No. 6,146,664 discloses a method of suspending ascorbic acid particulate in anhydrous silicone vehicles. The ascorbic acid particulate compositions are stable to moisture and air oxidation. High levels of particulate ascorbic acid can be suspended in the silicone vehicles (up to 40% by weight). For example, U.S. Pat. No. RE38,623 also discloses a method of suspending ascorbic acid particulate in anhydrous organic vehicles.

More recently, U.S. Pat. No. 6,361,783 discloses a method of dissolving ascorbic acid in nonaqueous polar organic solvents at high temperatures. Rapid cooling of the mixture to room temperature yields an ascorbic acid solution. Subsequently, the ascorbic acid solution serves as a disperse phase in an emulsion using a nonaqueous silicone vehicle as a continuous phase. However, the disclosed maximum solubility of ascorbic acid in dermatologically acceptable solvents such as, for example, glycerin or propylene glycol, is 17% by weight. It is highly desirable to achieve solubility levels of up to 40% by weight for maximum efficacy.

We have recently discovered that the presence of urea and/or urea derivatives, as solubilization enhancers, increases the amount of ascorbic acid dissolved in nonaqueous alcohol solvents when compared to the amount of ascorbic acid that would be soluble in the nonaqueous alcohol solvents in the absence of the solubilization enhancers. Concentrations of the dissolved ascorbic acid as high as 28% by weight have been achieved in the nonaqueous alcohol solvents utilizing urea and/or urea derivatives as the solubilization enhancers. This has been described in our co-pending application Ser. No. 11/242,306 of Oct. 3, 2005, which is incorporated herein by reference.

As indicated by these references, although ascorbic acid is important to the skin, it is difficult to formulate stabilized topical preparations (such as dermatological or cosmetic formulations), particularly at the higher concentrations needed for maximum efficacy, where ascorbic acid is dissolved in a dermatologically acceptable carrier, but not merely suspended. It is highly desirable to incorporate oleaginous skin protectants into the nonaqueous compositions comprising the dissolved ascorbic acid and solubilization enhancer in the alcohol solvents to further enhance stability and efficacy of the topical compositions, while maintaining the solubilization enhancing effect.

### SUMMARY OF THE INVENTION

Generally speaking, in accordance with the present invention, a method is provided for preparing a nonaqueous ascorbic acid composition in an alcohol solvent comprising the dissolved ascorbic acid, solubilization enhancer, and homogenized oleaginous skin protectant. A nonaqueous topical composition comprises the dissolved ascorbic acid, solubilization enhancer, and homogenized oleaginous skin protectant in the alcohol solvent.

Accordingly, it is an object of the invention to provide a method for preparing a nonaqueous ascorbic acid composition in the alcohol solvent comprising the dissolved ascorbic acid, solubilization enhancer, and homogenized oleaginous skin protectant.

Another object of the invention is to formulate a nonaqueous ascorbic acid topical composition, where the amount of dissolved ascorbic acid is effective to provide desirable anti-oxidative protection.

A further object of the invention is to incorporate the oleaginous skin protectant into the nonaqueous composition comprising the dissolved ascorbic acid and solubilization enhancer in the alcohol solvent for added stability and efficacy, while maintaining the solubilization enhancing effect.

Yet another object of the invention is to formulate a nonaqueous topical composition comprising the dissolved ascorbic acid, solubilization enhancer, and homogenized oleaginous skin protectant in the alcohol solvent.

Still other objects and advantages of the invention will, in part, be obvious and will, in part, be apparent from the following detailed description of the preferred embodiments.

### BRIEF DESCRIPTIONS OF THE DRAWINGS

FIG. 1 shows general structure of a urea derivative.

FIG. 2 shows general structure of a mono-substituted alkyl urea.

FIG. 3 shows general structure of a mono-substituted hydroxyalkyl urea.

### DETAILED DESCRIPTION OF THE PREFERRED EMBODIMENTS

In the disclosed embodiments, a method for preparing a nonaqueous ascorbic acid composition in an alcohol solvent comprising the dissolved ascorbic acid, solubilization enhancer, and homogenized oleaginous skin protectant, is described. A nonaqueous topical composition comprises the dissolved ascorbic acid, solubilization enhancer, and homogenized oleaginous skin protectant in the alcohol solvent.

The term ‘ascorbic acid’, when used in accordance with the present invention, means L-ascorbic acid, either synthetic or natural, the bio-available form, and derivatives thereof.

The term ‘dissolved’ or ‘dissolving’, as used herein, means that the ascorbic acid and/or solubilization enhancer is essentially solubilized in the nonaqueous alcohol solvent, and that the ascorbic acid and/or solubilization enhancer will not exist to any appreciable degree in the particulate or crystalline form.

The term ‘homogenized’ or ‘homogenizing’, as used herein, means that the oleaginous skin protectant is either solubilized or emulsified with the presence of a surfactant in the nonaqueous composition, depending on the nature of the oleaginous skin protectant and the alcohol solvent used in the composition.

The term ‘skin protectant’, when used in accordance with the present invention, is intended to denote substances that have beneficial or physiological (e.g., drug) effect on skin.

The term ‘safe and effective amount’, as used herein, means an amount of an oleaginous skin protectant used in the compositions and methods of the present invention, sufficient enough to significantly and positively modify the condition to be treated but low enough to avoid serious side effects, within the scope of sound medical advice.

A ‘solubilization enhancer’, as used herein, is an organic compound or a mixture of organic compounds that renders sparingly soluble substances more soluble in a solvent. An ‘effective’ amount of a solubilization enhancer means an amount sufficient enough to achieve desired solubilization enhancing effect in a nonaqueous composition of the present invention.

A ‘nonaqueous’ composition is one that is substantially water free. While water is not intentionally added to a nonaqueous composition, trace amounts of water (for example, existed in the solvent as an impurity) may still be present. It is desired that the amount of water in the nonaqueous composition be less than about 10% by weight, preferably less than 5% by weight, more preferably less than 3% by weight.

The term ‘room temperature’, as used herein, means a temperature of from about 18.degree. C. to about 25.degree. C.

The term ‘oleaginous’, as used herein, is interchangeable with the term ‘lipophilic’ or ‘hydrophobic’. The oleaginous compounds, as used herein, tend to be more soluble in nonpolar or weakly polar solvents such as, for example, oils, fats, ethers, esters, than in more polar solvents such as, for example, polyols.

In our co-pending application (Ser. No. 11/242,306 of Oct. 3, 2005), we have discovered that the presence of the solubilization enhancer increases the amount of ascorbic acid dissolved in the nonaqueous alcohol solvent when compared to the amount of ascorbic acid that would be soluble in the nonaqueous alcohol solvent in the absence of the solubilization enhancer.

In the present invention, we have unexpectedly discovered that through judicious selection of the alcohol solvents, surfactants, solubilization enhancers, and combinations thereof, and solubilization conditions, a safe and effective amount of the oleaginous skin protectants can be incorporated into the nonaqueous compositions, while maintaining the solubilization enhancing effect. The present invention will not only provide the benefits of maintaining the higher concentrations of dissolved ascorbic acid needed for maximum efficacy, but also enhance the beneficial and/or physiological effect of the composition by incorporating the oleaginous skin protectants.

Ascorbic acid is a well-known antioxidant of the general formula: C.sub.6H.sub.8 O.sub.6. The dissolved ascorbic acid in the nonaqueous composition delivers the anti-oxidative effect on the skin while being stable and effective. Solubilized topical compositions are generally more bio-available than compositions in which the active ingredient is insoluble or suspended.

The dissolved ascorbic acid may be present in an amount of at least 5% by weight, at least 10% by weight, or even as much as 40% by weight. Preferably dissolved ascorbic acid should be present in an amount of 10 to 35% by weight.

The organic solvents suitable for preparing the nonaqueous compositions of the present invention are alcohols having at least two carbon atoms. The general formula of the suitable alcohol solvents are: R(OH).sub.n where n is equal to or greater than 1 and R is generally C.sub.2-8 alkyl or substituted alkyl group. When n is equal to one, the alcohol is a monohydric alcohol. Examples of the suitable monohydric alcohol solvents are ethyl alcohol (also known as ethanol), 1-propanol, 2-propanol (also known as isopropanol), 1-butanol, 2-butanol, 1-pentanol, 2-pentanol, 3-pentanol, 1-hexanol, cyclohexanol, and combinations thereof.

It is known that certain monohydric alcohols, e.g., ethanol or isopropanol, are skin penetration and permeation enhancers. In addition to serving as a solvent, the presence of the monohydric alcohol in the nonaqueous alcohol carriers might also enhance delivery of the active ingredients across the skin barrier. Preferred monohydric alcohols are ethanol, isopropanol, and combinations thereof.

For purposes of this specification, polyols, also known as polyhydric alcohols, are defined as organic compounds having at least two hydroxyl groups per molecule. The general formula of the suitable polyols are: R(OH).sub.n where n is equal to or greater than 2 and R is generally C.sub.2-10 alkyl or substituted alkyl group.

Examples of the polyols suitable for preparing the nonaqueous compositions of the present invention are glycerin (also known as glycerol), propylene glycol (also known as 1,2-propanediol), 1,3-propanediol, 1,2-butanediol, 1,3-butanediol, 1,2-pentanediol, 1,5-pentanediol, 1,2-hexanediol, 1,6-hexanediol, diethylene glycol, diglycerin, dipropylene glycol, triethylene glycol, 1,2,3-hexanetriol, 1,2,6-hexanetriol, or combinations of the suitable polyols in any given ratio. 1,6-Hexanediol, also known as hexamethylene glycol, is a solid at room temperature (melting point: 42.8.degree. C.). In addition to serving as a nonaqueous alcohol solvent, it might also serve as a thickner to adjust the viscosity of the nonaqueous compositions. Preferred polyols are glycerin, propylene glycol, 1,5-pentanediol, 1,6-hexanediol, and combinations thereof.

Combinations of a monohydric alcohol and polyol in any given ratio are also suitable as the nonaqueous alcohol solvents. Polyols are especially preferred nonaqueous alcohol solvents.

The nonaqueous alcohol solvents may be present in an amount of 20 to 90% by weight, preferably 30 to 80% by weight, more preferably 40 to 70% by weight.

The solubilization enhancers of the present invention are organic compounds whose presence can increase the amount of ascorbic acid dissolved in the nonaqueous compositions when compared to the amount of ascorbic acid that would be soluble in the nonaqueous alcohol solvents in the absence of the solubilization enhancer. Preferably the solubilization enhancers are safe, chemically stable, dermatologically acceptable organic compounds, or combinations of such compounds. They should also be chemically compatible with other ingredients in the nonaqueous compositions.

We have found that urea and/or urea derivatives are the suitable solubilization enhancers. Urea, a diamide of carbonic acid, is a polar organic compound. Urea has the general formula of H.sub.2 N—C(O)—NH.sub.2. Urea is widely used as a moisturizing compound or keratolytic agent in cosmetic and dermatological applications.

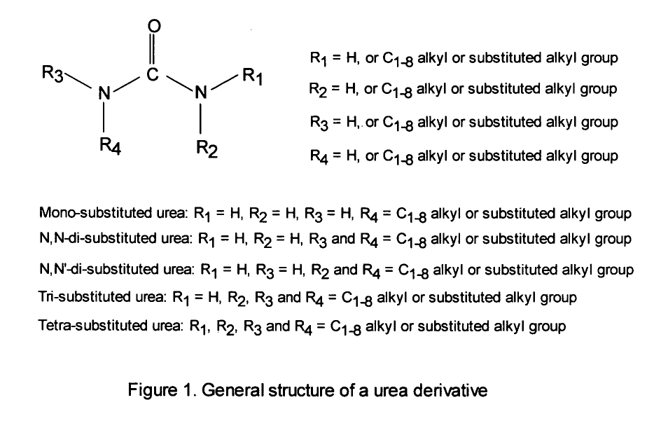

Urea derivatives are derived from urea by substituting one or more of the hydrogen atoms in the urea molecule with other suitable chemical groups. The urea derivatives, as used herein, have the general formula of R.sub.3 R.sub.4 N—C(O)—NR.sub.1 R.sub.2, where R.sub.1, R.sub.2, R.sub.3, and R.sub.4 are each independently hydrogen or C.sub.1-8 alkyl or substituted alkyl group. The alkyl group can be a straight or branched chain alkyl or a cycloalkyl group. Chemical groups such as, for example, hydroxyl, ether, can be substituted onto the alkyl group to give the substituted alkyl group. Examples of the suitable substituted alkyl group include, but not limited to, hydroxyalkyl group. The general structure of a urea derivative is shown in FIG. 1.

When one hydrogen atom in urea molecule is substituted by an alkyl or substituted alkyl group, a mono-substituted urea is formed. When two hydrogen atoms in urea molecule are substituted by alkyl or substituted alkyl groups, a di-substituted urea is formed. There are two types of di-substituted urea: N,N-di-substituted and N,N′-di-substituted urea. When three hydrogen atoms in urea molecule are substituted by alkyl or substituted alkyl groups, a tri-substituted urea is formed. When all four hydrogen atoms in urea molecule are substituted by alkyl or substituted alkyl groups, a tetra-substituted urea is formed.

Examples of the suitable di-substituted urea are N,N-dimethyl urea, N,N′-dimethyl urea, N,N-diethyl urea, N,N′-diethyl urea, N-ethyl-N′-2-hydroxyethyl urea, N,N′-bis-(1-butyl)urea, N,N′-bis-(2-hydroxyethyl)urea, N,N-bis-(2-hydroxyethyl)urea, N,N′-bis-(3-hydroxypropyl)urea, N,N-bis-(2-hydroxypropyl)urea, N,N′-bis-(2-hydroxypropyl)urea, and N,N′-bis-(4-hydroxybutyl)urea.

Examples of the suitable tri-substituted urea are N,N-bis-(2-hydroxypropyl)-N′-(2-hydroxyethyl)urea, N,N-bis-(2-hydroxyethyl)-N′-methyl urea, N,N-bis-(2-hydroxyethyl)-N′-ethyl urea, N,N-dimethyl-N′-(2-hydroxyethyl)urea, N,N-diethyl-N′-(2-hydroxyethyl)urea, and N,N-bis-(2-hydroxyethyl)-N′-propyl urea.

Examples of the suitable tetra-substituted urea are N,N,N′,N′-tetramethyl urea, N,N,N′,N′-tetrakis-(1-butyl)urea, N,N,N′,N′-tetrakis-(2-hydroxyethyl)urea, N,N,N′,N′-tetrakis-(2-hydroxypropyl)urea, and N,N-bis-(2-hydroxyethyl)-N′,N′-dimethyl urea.

Mono-substituted ureas are preferred solubilization enhancers. The mono-substituted ureas comprise mono-substituted alkyl and hydroxyalkyl ureas.

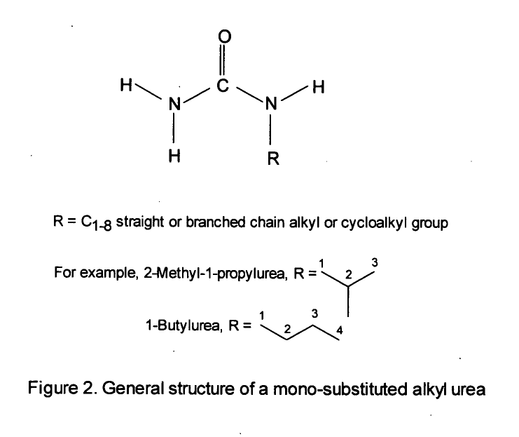

The mono-substituted alkyl ureas have the general structure as shown in FIG. 2. The alkyl group can be a straight or branched chain alkyl, or a cycloalkyl group. Examples of the suitable mono-substituted alkyl ureas are methyl urea, ethyl urea, 1-propyl urea, 2-propyl urea, 1-butyl urea, 2-butyl urea, 2-methyl-1-propyl urea, cyclohexyl urea, and combinations thereof.

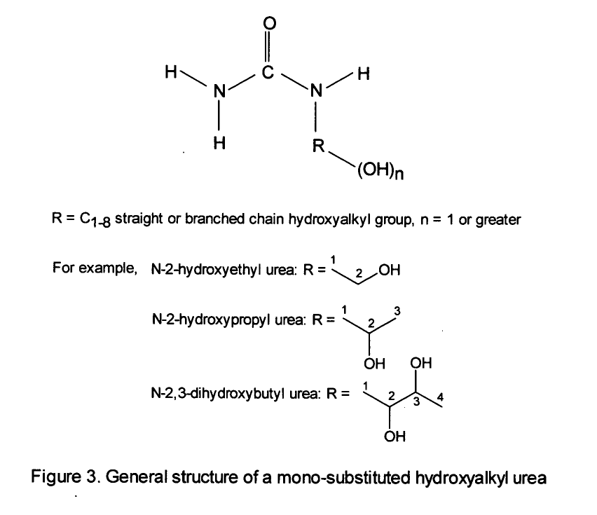

The mono-substituted hydroxyalkyl ureas have the general structure as shown in FIG. 3. The alkyl group can be a straight or branched chain alkyl group with one or more hydroxyl groups attached onto the alkyl chain at any suitable positions. Examples of the suitable mono-substituted hydroxyalkyl ureas are N-2-hydroxyethyl urea, N-3-hydroxypropyl urea, N-2-hydroxypropyl urea, N-2,3-dihydroxypropyl urea, N-4-hydroxybutyl urea, N-3-hydroxybutyl urea, N-2-hydroxybutyl urea, N-2,3-dihydroxybutyl urea, N-2,4-dihydroxybutyl urea, N-3,4-dihydroxybutyl urea, and combinations thereof.

Combinations of urea, the alkyl ureas, or hydroxyalkyl ureas in any given ratio are also suitable as the solubilization enhancers. The mono-substituted alkyl ureas are more hydrophobic than the parent compound urea due to the alkyl group. The hydrophobic property of the solubilization enhancers might be adjusted by varying the amount of the mono-substituted alkyl ureas being combined with urea. Especially preferred solubilization enhancer is urea.

Urea or the urea derivatives are polar molecules. With the exception of the tetra-substituted ureas, they have at least one hydrogen-bonding —N—H group per molecule. Ascorbic acid is also a polar molecule having multiple hydroxyl groups per molecule, which are capable of forming hydrogen bonds. Although the exact reason for the solubilization enhancing effect is unknown, without being bound by a particular theory, it is postulated that the hydrogen bonding and polar interactions are responsible for the surprising effect of the solubilization enhancement.

The dissolved urea or urea derivatives may be present in an amount of at least 1% by weight, at least 5% by weight, or even as much as 40% by weight, preferably 10 to 30% by weight.

Incorporation of oleaginous skin protectants into the nonaqueous compositions could have beneficial and/or physiological effect on skin, including but not limited to, lipid-soluble anti-oxidative, moisturizing, therapeutic, and cosmetic. The oleaginous skin protectants might be helpful in enhancing hydrophobic property of the nonaqueous compositions and reducing moisture absorption, thereby further improving stability and efficacy of the nonaqueous compositions.

In general, a polar organic compound such as, for example, ascorbic acid, tends to become less soluble in a polar organic solvent such as, for example, an alcohol solvent, when an oleaginous organic compound is added to the solvent mainly due to the increase in the hydrophobic property. However, through judicious selection of the nonaqueous alcohol solvents, surfactants, solubilization enhancers, and combinations thereof, and solubilization conditions, we have found that a safe and effective amount of the oleaginous skin protectants can be incorporated into the nonaqueous compositions of the present invention, while maintaining the solubilization enhancing effect.

Preferred oleaginous skin protectants useful in the present invention include, but not limited to the following groups of compounds, lipid-soluble antioxidants, moisturizing compounds, sunscreens, dermatologically active compounds, and aesthetic agents.

Preferred oleaginous antioxidants useful in the present invention include, but not limited to, tocopherols (vitamin E), tocopherol derivatives, tocotrienols, carotenoids, coenzymes Q, lipoic acid, vitamin A or derivatives, and ascorbic acid derivatives.

One antioxidant, vitamin E, is of particular interest. The term ‘vitamin E’ includes tocopherol (vitamin E) and derivatives thereof such as, for example, alpha.-, beta.-, .gamma.-, delta.-, epsilon.-, .zeta.sub.1, .zeta.sub.2, and eta.-tocopherol, and alpha.-tocopherol acetate, .alpha.-tocopherol nicotinate, .alpha.-tocopherol succinate, alpha.-tocopherol linoleate. Vitamin E is known as antioxidant and proactive vitamin for phospholipids of the cell membrane. It further has been known that vitamin E has a membrane-sealing effect. As with all antioxidants, vitamin E protects cells, including, epidermal cells which are susceptible to a wide range of oxidizing events.

Tocotrienols comprise one of the two groups of molecules belonging to vitamin E family, the other group being tocopherols. Examples of the suitable tocotrienols are alpha.-, beta.-, gamma.-, and delta.-, tocotrienol. The major sources of the tocotrienols are plant oils, such as, for example, palm oil, rice bran oil, and coconut oil.

Carotenoids are a class of natural fat-soluble pigments. The majority carotenoids are derived from a 40-carbon polyene chain. Examples of the suitable carotenoids are beta-carotene and lutein.

The coenzymes Q (also known as ubiquinones) are naturally occurring in the majority of aerobic organisms, from bacteria to higher plants and animals, and define a group of lipid-soluble benzoquinones involved in electron transport in mitochondria. The ubiquinones all share the 2,3-dimethoxy-5-methyl-benzoquinone nucleus but differ in terms of the terpenoid side chain. The terpenoid side chain comprises from 1 to 12 mono-unsaturated trans isoprene units. Coenzyme Q10, having a terpenoid side chain of 10 isoprene units, is one of the coenzymes Q. It is the most common of the coenzymes Q. Coenzyme Q10 is an effective lipid-soluble antioxidant. It is believed that coenzyme Q10 protects skin against the damaging effect of free radicals, particularly the lipid peroxidation. The reduced form of ubiquinone, ubiquinol, is also an effective lipid-soluble antioxidant. The nonaqueous composition may contain coenzyme Q10, or any other ubiquinone, or ubiquinol, or combinations thereof.

Lipoic acid, also known as thioctic acid, .alpha.-lipoic acid, is a coenzyme for pyruvate dehydrogenase and .alpha.-ketoglutarate dehydrogenase in mitochondria. Either the naturally occurring D-lipoic acid or racemic mixture of the DL-lipoic acids is suitable for use in the present invention. Free lipoic acid is rapidly taken up by cells and reduced to dihydrolipoic acid (DHLA) intracellularly. DHLA, the reduced form of lipoic acid, is a potent antioxidant that can regenerate other antioxidants, such as vitamins C, E and coenzyme Q10.

The term ‘vitamin A’ includes retinol and derivatives thereof such as, for example, retinyl palmitate, retinyl acetate, or other esters formed between retinol and a carboxylic acid, or retinoic acids such as, for example, tretinoin, and isotretinoin.

Examples of the oleaginous ascorbic acid derivatives are ascorbic acid alkanoates. Ascorbic acid alkanoates are esters formed between ascorbic acid and fatty acids. The ascorbic acid alkanoate can be a mono-ester, di-ester, or tri-ester depending on the number of the hydroxyl groups in ascorbic acid being esterified to the fatty acids. The mono-ester has the general formula of ascorbyl—O—C(O)—[CH.sub.2].sub.n —CH.sub.3, where n is generally from 8 to 20. Examples of the suitable ascorbic acid mono-ester are ascorbyl palmitate, ascorbyl laurate, ascorbyl myristate, and ascorbyl stearate. Examples of the suitable ascorbic acid di-ester are ascorbyl dipalmitate and ascorbyl distearate. Examples of the suitable ascorbic acid tri-ester are ascorbyl tripalmitate and ascorbyl tristearate.

Incorporation of the oleaginous antioxidants into the nonaqueous compositions might provide additional lipid-soluble anti-oxidative protection. It is believed that combinations of the oleaginous antioxidants and dissolved ascorbic acid might provide a synergistic anti-oxidative effect for maximum protection.

Preferred oleaginous moisturizing compounds useful in the present invention include, but not limited to, petrolatum, squalane, hydrogenated polydecene, isododecane, isohexadecane, mineral oils, vegetable oils and waxes, synthetic waxes, emollient esters, lanolins and their derivatives, unsaturated fatty acids and their derivatives. Examples of the suitable vegetable oils and waxes are apricot kernel oil, avocado oil, canola oil, olive oil, sesame oil, sweet almond oil, peanut oil, rapeseed oil, safflower oil, sunflower oil, beeswax, candelilla wax, carnauba wax, shea butter, jojoba oil, and so on. Examples of the suitable synthetic waxes are synthetic beeswax, synthetic candelilla wax, synthetic carnauba wax, synthetic japan wax, synthetic jojoba oil, and so on. Examples of the suitable emollient esters are caprylic/capric triglycerides, stearyl stearate, isopropyl myristate, isopropyl palmitate, isopropyl isostearate, cetyl esters, and C12-13 alkyl lactate. Particularly suitable moisturizing compounds are petrolatum, squalane, hydrogenated polydecene, mineral oils, vegetable oils and waxes, and emollient esters.

Preferred oleaginous sunscreen compounds useful in the present invention include, but not limited to, organic UV filters. Examples of the suitable organic UV filters are 2-ethylhexyl-3-cyano-3,3-diphenyl-2-propenoate (octocrylene), butyl methoxydibenzoylmethane (avobenzene), 2-ethylhexyl methoxycinnamate (octyl methoxycinnamate), oxybenzone (benzophenone-3), sulisobenzone (benzophenone-4), dioxybenzone (benzophenone-8), and 2-ethylhexyl salicylate (octyl salicylate). The oleaginous UV filters can add UV protection benefits to the nonaqueous compositions of the present invention.

Preferred oleaginous dermatologically active agents useful in the present invention include, but not limited to, skin whitening agents, anti-bacterial agents, anti-fungal agents, and anti-inflammatory agents. Examples of the suitable skin whitening agents are hydroquinone and 4-methoxyphenol. Examples of the suitable anti-bacterial agents are bacitracin, neomycin, erythromycin, and mupirocin. Examples of the suitable anti-fungal agents are clotrimazole, ketoconazole, and miconazole. Examples of the suitable anti-inflammatory agents are topical corticosteroids such as, for example, diflorasone diacetate, betamethasone valerate, and clobetosol propinonate.

Preferred oleaginous aesthetic agents useful in the present invention include, but not limited to, silicone oils. Silicone oils refer to organosiloxanes or polyorganosiloxanes, which are any of a large group of siloxane polymers. Examples of the suitable silicone oils are polysilicone-11, dimethicone, and cyclomethicone. Added alone or in combination with other oleaginous skin protectants, the aesthetic agents might improve skin feel of the nonaqueous compositions of the present invention.

The safe and effective amount of the oleaginous skin protectants of the present invention will vary with the particular protectant and the nature and duration of treatment. For example, vitamin A and derivatives may be present in an amount of at least 0.01% by weight, at least 0.1% by weight, or even as much as 2% weight. For example, vitamin E and derivatives may be present in an amount of at least 0.1% by weight, at least 5% by weight, or even as much as 40% by weight. For example, ascorbic acid alkanoates might be present in an amount of at least 0.1% by weight, as much as 5% by weight, or even as much as 20% by weight. For example, coenzyme Q10 may be present in an amount of at least 0.01% by weight, at least 1% by weight, or even as much as 5% by weight. Lipoic acid may be present in an amount of at least 0.05% by weight, at least 1% by weight, or even as much as 10% by weight. For example, the moisturizing compounds or aesthetic agents may be present in an amount of at least 1% by weight, at least 10% by weight, or even as much as 50% by weight. For example, the dermatologically active compounds may be present in an amount of at least 0.01% by weight, at least by 0.05% by weight, or even as much as 15% by weight. For example, the compositions of the present invention may contain 0.01 to 20% by weight, more preferably 0.05 to 10% by weight of one or more the oleaginous sunscreens.

The compositions may contain 0.1 to 15%, preferably 0.5 to 10% by weight of the total composition of one or more surfactants. The term ‘surfactant’ is defined, in accordance with the present invention, as a compound having at least one hydrophilic moiety and at least one lipophilic moiety. The surfactants may be silicone surfactants (also referred to as organosiloxane emulsifiers) or organic surfactants or combinations of the silicone surfactants and organic surfactants.

The silicone emulsifier is a polymer containing a polymeric backbone including repeating siloxy units, for example, di-alkylsiloxy units, preferably dimethylsiloxy units. The hydrophilic portion of the organosiloxane is generally a hydrophilic chemical group substituted onto the polymer backbone. The repeating dimethylsiloxy units of the emulsifier are lipophilic in nature due to the methyl groups. Examples of the suitable silicone emulsifiers are cetyl dimethicone copolyol and dimethicone copolyol.

Also suitable as surfactants are various organic surfactants such as anionic, nonionic, amphoteric, zwitterionic, or cationic surfactants. Preferred surfactants are anionic and nonionic.

Anionic surfactants include alkyl and alkyl ether sulfates where alkyl groups have from about 10 to 20 carbon atoms. Examples of the suitable alkyl and alkyl ether sulfates are sodium lauryl sulfate and sodium laureth sulfate. Other class of anionic surfactants includes N-acyl amino surfactants and salts thereof. Examples of such surfactants are N-lauroyl sarcosinate, N-myristoyl sarcosinate, N-cocoyl sarcosinate, preferably in sodium forms. Nonionic surfactants are generally compounds formed by the condensation of alkylene oxide groups such as, for example, ethylene oxide and propylene oxide, with a lipophilic compound. Examples of classes of nonionic surfactants are:

(a). Polysorbates, or sorbitol or sucrose esters of fatty acids. Examples of the suitable polysorbates are polysorbate-20, polysorbate-40, polysorbate-60, polysorbate-65, polysorbate-80, and polysorbate-85. Examples of the suitable sorbitol esters of fatty acids are sorbitan monooleate, sorbitan monopalmitate, sorbitan monolaurate, sorbitan monostearate, sorbitan monoisostearate, sorbitan sesquioleate, sorbitan trioleate, sorbitan tristearate, sorbitan monooleate ethoxylate EO 20 mole, sorbitan monolaurate ethoxylate EO 20 mole, sorbitan monopalmitate ethoxylate EO 20 mole, sorbitan monostearate ethoxylate EO 20 mole, and so on.

(b). Alkyl polysaccharides having a lipophilic group of 6 to 30 carbon atoms and polysaccharide group such as glucose, galactose, and so on. Examples of the suitable alkyl polysaccharides are octyl, nonydecyl, undecyl, dodecyl, hexadecyl, octadecyl gluocosides, galactosides, and so on.

(c). Polyol or polyethylene glycol (PEG) esters of fatty acids and PEG ethers of fatty alcohols. Examples of the suitable fatty esters or ethers are glyceryl stearate, glyceryl distearate, PEG-40 stearate, PEG-50 stearate, PEG-100 stearate, oleth-5, oleth-10, oleth-20, laureth-23, ceteareth-20, ceteareth-21, steareth-10, steareth-21, and so on.

Combinations of the surfactants from each category are also suitable as the surfactants to be used in the nonaqueous compositions. Especially preferred surfactants are organic nonionic surfactants and combinations thereof.

It may also be desired to include certain other ingredients in the nonaqueous compositions of the present invention such as cholesterol, phospholipids, ceramides, fatty acids and alcohols, viscosity modifiers, and so on.

Examples of the suitable phospholipids are phosphatidyl ethanolamine and phosphatidyl choline. Phospholipids are diglycerides that are covalently bonded to a phosphate group by an ester linkage. The diglyceride is composed of a glycerol backbone that has esterified to two fatty acids. Cell membranes are composed of two layers of phospholipids in a bilayer arrangement. Ceramides consist of a sphingoid base (a long chain aliphatic amine, containing two or three hydroxyl groups) linked to a fatty acid via an amide bond. Although rarely found as such at greater than trace levels in tissues, they can exert important biological effects. Examples of the suitable ceramides are ceramide I, ceramide III, ceramide IIIA, Ceramide IIIB, and ceramide VI. Ceramides are available from a number of suppliers such as, for example, Centerchem, Inc. (Norwalk, Conn.).

Ceramides or phospholipids may be present in an amount of at least 0.05% by weight, at least 0.5% by weight, or even as much as 5% by weight.

Examples of the suitable fatty acids or alcohols are arachidic acid, behenic acid, capric acid, caprylic acid, lauric acid, myristic acid, linoleic acid, linolenic acid, oleic acid, stearic acid, palmitic acid, cetearyl alcohol, cetyl alcohol, stearyl alcohol, oleyl alcohol, and so on.

Fatty acids or alcohols may be present in an amount of at least 0.1% by weight, at least 1% by weight, or even as much as 10% by weight.

Cholesterol may be present in an amount of at least 0.05% by weight, at least 0.5% by weight, or even as much as 5% by weight.

Cholesterol, ceramides and fatty acids are the three main lipids in skin barrier. Incorporation of these ingredients into the nonaqueous compositions of the present invention might help restoration of damaged skin barrier.

Examples of the suitable viscosity modifiers are ethoxydiglycol, the carbomer or Carbopol. Ethoxydiglycol, which is mono ethyl ether of diethylene glycol, is commonly used in cosmetic preparations to reduce viscosity. For example, ethoxydiglycol may be present in an amount of 1 to 10% by weight. The carbomer or Carbopol are resins which are known thickening agents. Examples of the suitable carbomer or Carbopol are Carbopol 934, 940, 941, Ultrez 10, and Ultrez 20. The carbomer or Carbopol may be present in amount of about 0.05% to about 3% by weight.

It has been discovered in the present invention that a safe and effective amount of the oleaginous skin protectants can be incorporated into the nonaqueous compositions, while maintaining the solubilization enhancing effect. Most surprisingly, concentrations of the dissolved ascorbic acid as high as 25% by weight have been achieved in the nonaqueous compositions comprising a safe and effective amount of the oleaginous skin protectant.

The solubilization enhancing effect has been observed when the nonaqueous compositions of the present invention are prepared at a temperature higher than room temperature. Yet another unexpected discovery of the present invention is that the solubilization enhancing effect has also been observed even when the nonaqueous compositions of the present invention are prepared at room temperature.

The solubilization process might be conducted at any temperature from room temperature up to just below the boiling point of the alcohol solvent used in the nonaqueous composition. The preferred temperature for conducting the solubilization process is a temperature of from about 50.degree. C. to about 120.degree. C., more preferably from about 60.degree. C. to about 100.degree. C.

In accordance with the present invention, the preferred method for preparing nonaqueous ascorbic acid compositions in the presence of the solubilization enhancers and oleaginous skin protectants are as follows:
subjecting the nonaqueous alcohol solvent to a temperature of from room temperature to about 160.degree. C., preferably to a temperature of from about 50 to about 120.degree. C., more preferably from about 60.degree. C. to about 100.degree. C.,
adding the solubilization enhancer and the ascorbic acid together to the solvent while maintaining the desirable temperature, while stirring until dissolved to yield a mixture of the dissolved ascorbic acid and solubilization enhancer,
adding a safe and effective amount of the oleaginous skin protectant and an optional surfactant to the mixture,
homogenizing the mixture,
cooling the mixture to room temperature if the solubilization process is conducted at a temperature higher than room temperature.

An alternative method of preparation, where the solubilization enhancer is dissolved first, is described as follows:
subjecting the nonaqueous alcohol solvent to a temperature of from room temperature to about 160.degree. C., preferably to a temperature of from about 50 to about 120.degree. C., more preferably from about 60.degree. C. to about 100.degree. C.,
adding the solubilization enhancer while maintaining the desirable temperature, while stirring until dissolved,
adding the ascorbic acid to the mixture while maintaining the desirable temperature, while stirring until dissolved,
adding a safe and effective amount of the oleaginous skin protectant and an optional surfactant to the mixture,
homogenizing the mixture,
cooling the mixture to room temperature if the solubilization process is conducted at a temperature higher than room temperature.

If the solubilization is conducted at a temperature higher than room temperature, yet another alternative method of preparation is that the solubilization enhancer and ascorbic acid can be added to the alcohol solvent at room temperature. Then, the alcohol solvent, solubilization enhancer, and ascorbic acid can be heated together to the desirable temperature, for example, a temperature of from about 30 to about 160.degree. C., preferably to a temperature of from about 50 to about 120.degree. C., more preferably from about 60.degree. C. to about 100.degree. C., while stirring until dissolved. Then, a safe and effective amount of the oleaginous skin protectant and an optional surfactant can be added to the mixture. After completely homogenized, the mixture is allowed to cool to room temperature.

The following examples are included for purposes of illustrating the technology covered by this disclosure. They are not intended to be exhaustive or to limit the scope of the claimed invention in any manner. One skilled in the art will understand that there are alternatives to these specific embodiments that are not completely described by these examples.

### EXAMPLE 1 (FOR COMPARISON)

This example is to demonstrate that ascorbic acid has low solubility in nonaqueous propylene glycol in the absence of a solubilization enhancer.

| Component | Amount (weight percentage) |
|---|---|
| Propylene glycol | 80% |
| L-Ascorbic acid | 20% |

The propylene glycol was heated to 80.degree. C. The ascorbic acid was added to the propylene glycol at 80.degree. C. The mixture was maintained at 80.degree. C., while stirring for a minimum of 5 hours. The ascorbic acid was not completely dissolved in the propylene glycol under the experimental condition.

It was also determined that in a composition comprising, by weight, 17% ascorbic acid and 83% propylene glycol, prepared by heating the mixture at 80.degree. C., the ascorbic acid was soluble under this experimental condition. This result is consistent with the solubility data disclosed in U.S. Pat. No. 6,361,783, EXAMPLE 4.

### EXAMPLE 2

This example is to demonstrate the solubilization enhancing effect of urea in propylene glycol. A nonaqueous composition in accordance with the invention was prepared as follows:

| Component | Amount (weight percentage) |
|---|---|
| Propylene glycol | 50% |
| Urea | 22% |
| L-Ascorbic acid | 28% |

The propylene glycol was heated to 80.degree. C. The urea was added to the propylene glycol while maintaining the temperature at 80.degree. C., while stirring until dissolved. Then, ascorbic acid was added to the mixture while maintaining the temperature at 80.degree. C., while stirring until dissolved. It took less than 4 hours for the ascorbic acid and solubilization enhancer to dissolve completely. The mixture was allowed to cool to room temperature. The mixture was a clear solution.

This example demonstrated that in the presence of urea as the solubilization enhancer, as high as 28% of ascorbic acid (by weight) can be dissolved in the nonaqueous polyol solvent.

### EXAMPLE 3 (FOR COMPARISON)

This example is to provide a comparative reference for demonstrating the solubilization enhancing effect of urea in monohydric alcohols. Absolute ethanol, which is 100% ethanol, was used as the nonaqueous solvent.

| Component | Amount (weight percentage) |
|---|---|
| Ethanol (absolute) | 97.50% |
| L-Ascorbic acid | 2.50% |

The solubilization process was conducted at room temperature. The ascorbic acid was added to the ethanol at room temperature. After stirring for about 36 hours at room temperature, the ascorbic acid was still not completely dissolved.

### EXAMPLE 4

This example is to demonstrate the solubilization enhancing effect of urea in ethanol, a monohydric alcohol. A nonaqueous composition in accordance with the invention was prepared as follows:

| Component | Amount (weiaht percentage) |
|---|---|
| Ethanol (absolute) | 95.50% |
| Urea | 2.00% |
| L-Ascorbic acid | 2.50% |

The experimental condition here was same as that in EXAMPLE 3. The two sets of experiments (EXAMPLE 3 and EXAMPLE 4) were carried out side by side for comparison.

The solubilization process was conducted at room temperature. The ascorbic acid and urea were added to the ethanol at room temperature. After stirring for about 16 hours at room temperature, the ascorbic acid and urea were completely dissolved.

This example demonstrated the solubilization enhancing effect in the nonaqueous monohydric alcohol solvent.

### EXAMPLE 5 (FOR COMPARISON)

This example is to provide a comparative reference for demonstrating the solubilization enhancing effect of urea in the polyol at room temperature. Nonaqueous propylene glycol was used as the solvent:

| Component | Amount (weight percentage) |
|---|---|
| Propylene glycol | 94.5% |
| L-Ascorbic acid | 5.5% |

The solubilization process was carried out at room temperature. The ascorbic acid was added to the propylene glycol. After stirring for about 16 hours at room temperature, the ascorbic acid (at 5.5% by weight) was still not completely dissolved.

Solubility of ascorbic acid in propylene glycol is reported to be about 4.6% by weight (The Merck Index, 11^{th} Edition, entry 855). Thus, this example demonstrated that in the absence of a solubilization enhancer, 5.5% by weight of ascorbic acid can not be dissolved in propylene glycol at room temperature.

### EXAMPLE 6

This example is to demonstrate the solubilization enhancing effect of urea in propylene glycol when the solubilization process is conducted at room temperature. A nonaqueous composition in accordance with the invention was prepared as follows:

| Component | Amount (weight percentage) |
|---|---|
| Propylene glycol | 88.0% |
| Urea | 5.0% |
| L-Ascorbic acid | 7.0% |

The experimental condition here was same as that in EXAMPLE 5. The two sets of experiments (EXAMPLE 5 and EXAMPLE 6) were carried out side by side for comparison.

The ascorbic acid and urea were added to the propylene glycol at room temperature. After stirring for about 16 hours at room temperature, the ascorbic acid (at 7% by weight) and urea were completely dissolved.

This example demonstrated the solubilization enhancing effect of urea in the nonaqueous polyol solvent when the solubilization process is conducted at room temperature.

### EXAMPLE 7

This example is to formulate a nonaqueous ascorbic acid composition comprising urea as the solubilization enhancer and vitamin E acetate as the oleaginous skin protectant in ethanol at room temperature. A nonaqueous composition in accordance with the invention was prepared as follows:

| Component | Amount (weight percentage) |
|---|---|
| Ethanol (absolute) | 94.50% |
| Urea | 2.00% |
| L-Ascorbic acid | 2.50% |
| Vitamin E acetate | 1.00% |

The urea and ascorbic acid were added together to the ethanol at room temperature. The mixture was kept at room temperature, while stirring until dissolved. Vitamin E acetate was added to the mixture. Vitamin E acetate was solubilized in the mixture. No surfactant is needed. The mixture was a clear solution.

### EXAMPLE 8

This example is to formulate a nonaqueous ascorbic acid composition comprising urea as the solubilization enhancer and vitamin E acetate as the oleaginous skin protectant in propylene glycol at room temperature. A nonaqueous composition in accordance with the invention was prepared as follows:

| Component | Amount (weight percentage) |
|---|---|
| Propylene glycol | 84.0% |
| Urea | 5.0% |
| L-Ascorbic acid | 6.5% |
| Polysorbate-80 | 1.5% |
| Vitamin E acetate | 3.0% |

The urea and ascorbic acid were added together to the propylene glycol at room temperature. The mixture was kept at room temperature, while stirring until the ascorbic acid and solubilization enhancer were dissolved. Polysorbate-80 and vitamin E acetate were added to the mixture. The mixture was stirred vigorously to allow it homogenized. The mixture was an opaque solution.

### EXAMPLE 9

This example is to formulate a nonaqueous ascorbic acid composition comprising urea as the solubilization enhancer and vitamin E acetate as the oleaginous skin protectant in propylene glycol. Polysorbate-80 was chosen as a nonionic surfactant. As an alternative method for conducting the solubilization process, the solubilization enhancer was first dissolved in the polyol solvent before the ascorbic acid was added to the mixture. A nonaqueous composition in accordance with the invention was prepared as follows:

| Component | Amount (weight percentage) |
|---|---|
| Propylene glycol | 55% |
| Urea | 20% |
| L-Ascorbic acid | 22% |
| Polysorbate-80 | 2% |
| Vitamin E acetate | 1% |

The propylene glycol was heated to 75.degree. C. The urea was added to the propylene glycol while maintaining the temperature at 75.degree. C., while stirring until dissolved. Then, the ascorbic acid was added to the mixture while maintaining the temperature at 75.degree. C., while stirring until dissolved. Polysorbate-80 and vitamin E acetate were added to the mixture. The mixture was stirred vigorously to allow it homogenized. Then, the mixture was allowed to cool to room temperature. The mixture was an opaque solution.

### EXAMPLE 10

This example is to formulate a nonaqueous ascorbic acid composition comprising urea as the solubilization enhancer and vitamin E acetate as the oleaginous skin protectant in the propylene glycol. A higher concentration of vitamin E acetate at 8% was used to prepare a more lipophilic product. Polysorbate-80 was chosen as a nonionic surfactant. As an alternative method for conducting the solubilization process, the solubilization enhancer was first dissolved in the polyol solvent before the ascorbic acid was added to the mixture. A nonaqueous composition in accordance with the invention was prepared as follows:

| Component | Amount (weight percentage) |
|---|---|
| Propylene glycol | 52% |
| Urea | 15% |
| L-Ascorbic acid | 20% |
| Polysorbate-80 | 5% |
| Vitamin E acetate | 8% |

The propylene glycol was heated to 75.degree. C. The urea was added to the propylene glycol while maintaining the temperature at 75.degree. C., while stirring until dissolved. Then, the ascorbic acid was added to the mixture while maintaining the temperature at 75.degree. C., while stirring until dissolved. Polysorbate-80 and vitamin E acetate were added to the mixture. The mixture was stirred vigorously to allow it homogenized. Then, the mixture was allowed to cool to room temperature. The mixture was an opaque solution.

### EXAMPLE 111

This example is to formulate a nonaqueous ascorbic acid composition comprising urea as the solubilization enhancer and vitamin E acetate as the oleaginous skin protectant in a mixture of the polyols, propylene glycol and 1,6-hexanediol. An even higher concentration of vitamin E acetate at 10% was used. Polysorbate-80 was chosen as a nonionic surfactant. A nonaqueous composition in accordance with the invention was prepared as follows:

| Component | Amount (weight percentage) |
|---|---|
| Propylene glycol | 48% |
| 1,6-Hexanediol | 5% |
| Urea | 12% |
| L-Ascorbic acid | 20% |
| Polysorbate-80 | 5% |
| Vitamin E acetate | 10% |

The mixture of propylene glycol and 1,6-hexanediol was heated to 75.degree. C. The urea and ascorbic acid were added together to the propylene glycol/1,6-hexanediol while maintaining the temperature at 75.degree. C., while stirring until dissolved. Polysorbate-80 and vitamin E acetate were added to the mixture. The mixture was stirred vigorously to allow it homogenized. Then, the mixture was allowed to cool to room temperature. The mixture was an opaque solution.

### EXAMPLE 12

This example is to formulate a nonaqueous ascorbic acid composition comprising urea as the solubilization enhancer and vitamin E acetate as the oleaginous skin protectant in propylene glycol. Vitamin E acetate at 10% was used to prepare a more lipophilic product. Higher concentration of vitamin E acetate could also provide better anti-oxidative protection. A nonaqueous composition in accordance with the invention was prepared as follows:

| Component | Amount (weight percentage) |
|---|---|
| Propylene glycol | 52% |
| Urea | 12% |
| L-Ascorbic acid | 20% |
| Polysorbate-80 | 4% |
| Oleth-20 | 2% |
| Vitamin E acetate | 10% |

The propylene glycol was heated to 75.degree. C. The urea was added to the propylene glycol while maintaining the temperature at 75.degree. C., while stirring until dissolved. Then, the ascorbic acid was added to the mixture while maintaining the temperature at 75.degree. C., while stirring until dissolved. Oleth-20, polysorbate-80, and vitamin E acetate were added to the mixture. The mixture was stirred vigorously to allow it homogenized. Then, the mixture was allowed to cool to room temperature. The mixture was an opaque solution.

### EXAMPLE 13

This example is to formulate a nonaqueous ascorbic acid composition comprising urea as the solubilization enhancer and a moisturizing vegetable oil, safflower oil, as the oleaginous skin protectant in propylene glycol. A nonaqueous composition in accordance with the invention was prepared as follows:

| Component | Amount (weight percentage) |
|---|---|
| Propylene glycol | 52% |
| Urea | 12% |
| L-Ascorbic acid | 20% |
| Oleth-20 | 3% |
| Polysorbate-80 | 3% |
| Safflower oil | 10% |

The propylene glycol was heated to 75.degree. C. The urea and ascorbic acid were added together to the propylene glycol while maintaining the temperature at 75.degree. C., while stirring until dissolved. Oleth-20, polysorbate-80, and safflower oil were added to the mixture. The mixture was stirred vigorously to allow it homogenized. Then, the mixture was allowed to cool to room temperature. The mixture was an opaque solution.

### EXAMPLE 14

This example is to formulate a nonaqueous ascorbic acid composition comprising urea as the solubilization enhancer and silicone oil as the oleaginous skin protectant in propylene glycol. The silicone oil, dimethicone, might provide better smooth feel for the sample. A nonaqueous composition in accordance with the invention was prepared as follows:

| Component | Amount (weight percentage) |
|---|---|
| Propylene glycol | 60% |
| Urea | 12% |
| L-Ascorbic acid | 20% |
| Oleth-20 | 1% |
| Polysorbate-80 | 2% |
| Dimethicone (ISP Corp. DM100) | 5% |

The propylene glycol was heated to 75.degree. C. The urea and ascorbic acid were added together to the propylene glycol while maintaining the temperature at 75.degree. C., while stirring until dissolved. Oleth-20, polysorbate-80, and dimethicone were added to the mixture. The mixture was stirred vigorously to allow it homogenized. Then, the mixture was allowed to cool to room temperature. The mixture was an opaque gel. Incorporation of the silicone enhanced the perception of smooth skin feel.

### EXAMPLE 15

This example is to formulate a nonaqueous ascorbic acid composition comprising a mixture of urea and 1-butyl urea as the solubilization enhancer and vitamin E acetate as the oleaginous skin protectant in the propylene glycol. 1-Butyl urea is more lipophilic than urea due to the butyl group, which might be helpful in solubilizing the oleaginous skin protectant. 1-Butyl urea is available from Lancaster Synthesis (Ward Hill, Mass.). A nonaqueous composition in accordance with the invention was prepared as follows:

| Component | Amount (weight percentage) |
|---|---|
| Propylene glycol | 50% |
| Urea | 12% |
| 1-Butyl urea | 5% |
| L-Ascorbic acid | 20% |
| Polysorbate-80 | 3% |
| Oleth-20 | 2% |
| Vitamin E acetate | 8% |

The propylene glycol was heated to 75.degree. C. The urea, 1-butyl urea, and ascorbic acid were added together to the propylene glycol while maintaining the temperature at 75.degree. C., while stirring until dissolved. Polysorbate-80, oleth-20, and vitamin E acetate were added to the mixture. The mixture was stirred vigorously to allow it homogenized. Then, the mixture was allowed to cool to room temperature. The mixture was an opaque gel.

### EXAMPLE 16

This example is to formulate a nonaqueous ascorbic acid composition comprising urea as the solubilization enhancer and vitamin E acetate as the oleaginous skin protectant in the propylene glycol. Incorporation of a moisturizing emollient ester, caprylic/capric triglyceride. into the composition was intended to enhance moisturizing property and skin feel of the sample. A nonaqueous composition in accordance with the invention was prepared as follows:

| Component | Amount (weight percentage) |
|---|---|
| Propylene glycol | 50% |
| Urea | 12% |
| L-Ascorbic acid | 20% |
| Polysorbate-80 | 3% |
| Oleth-20 | 3% |
| Vitamin E acetate | 8% |
| Caprylic/capric triglyceride | 4% |

The propylene glycol was heated to 75.degree. C. The urea and ascorbic acid were added together to the propylene glycol while maintaining the temperature at 75.degree. C., while stirring until dissolved. Polysorbate-80, oleth-20, caprylic/capric triglyceride, and vitamin E acetate were added to the mixture. The mixture was stirred vigorously to allow it homogenized. Then, the mixture was allowed to cool to room temperature. The mixture was an opaque solution.

### EXAMPLE 17

This example is to formulate a nonaqueous ascorbic acid composition comprising urea as the solubilization enhancer and an organic sunscreen as the oleaginous skin protectant in the propylene glycol. Octocrylene is available from DSM Nutritional Products (Parsippany, N.J.). A nonaqueous composition in accordance with the invention was prepared as follows:

| Component | Amount (weight percentage) |
|---|---|
| Propylene glycol | 55.5% |
| Urea | 12% |
| L-Ascorbic acid | 20% |
| Polysorbate-80 | 3% |
| Oleth-20 | 2% |
| Octocrylene | 7.5% |

The propylene glycol was heated to 75.degree. C. The urea and ascorbic acid were added together to the propylene glycol while maintaining the temperature at 75.degree. C., while stirring until dissolved. Polysorbate-80, oleth-20, and octocrylene were added to the mixture. The mixture was stirred vigorously to allow it homogenized. Then, the mixture was allowed to cool to room temperature. The mixture was an opaque gel.

### EXAMPLE 18

This example is to formulate a nonaqueous ascorbic acid composition comprising urea as the solubilization enhancer and vitamin E acetate as the oleaginous skin protectant in the propylene glycol. Silicone oil, dimethicone, was added to improve aesthetic property of the sample. A nonaqueous composition in accordance with the invention was prepared as follows:

| Component | Amount (weight percentage) |
|---|---|
| Propylene glycol | 52% |
| Urea | 12% |
| L-Ascorbic acid | 20% |
| Polysorbate-80 | 3% |
| Oleth-20 | 3% |
| Vitamin E acetate | 8% |
| Dimethicone (ISP Corp. DM100) | 2% |

The propylene glycol was heated to 75.degree. C. The urea and ascorbic acid were added together to the propylene glycol while maintaining the temperature at 75.degree. C., while stirring until dissolved. Polysorbate-80, oleth-20, dimethicone, and vitamin E acetate were added to the mixture. The mixture was stirred vigorously to allow it homogenized. Then, the mixture was allowed to cool to room temperature. The mixture was an opaque gel.

### EXAMPLE 19

This example is to formulate a nonaqueous ascorbic acid composition comprising urea as the solubilization enhancer and vitamin E acetate as the oleaginous skin protectant in the propylene glycol. A lipid-soluble ascorbic acid alkanoate, ascorbyl palmitate, was incorporated into the composition. Ascorbyl palmitate is available from DSM Nutritional Products (Parsippany, N.J.). A nonaqueous composition in accordance with the invention was prepared as follows:

| Component | Amount (weight percentage) |
|---|---|
| Propylene glycol | 52% |
| Urea | 12% |
| L-Ascorbic acid | 20% |
| L-ascorbyl palmitate | 3% |
| Polysorbate-80 | 4% |
| Oleth-20 | 1% |
| Vitamin E acetate | 8% |

The propylene glycol was heated to 75.degree. C. The urea and ascorbic acid were added together to the propylene glycol while maintaining the temperature at 75.degree. C., while stirring until dissolved. Polysorbate-80, oleth-20, ascorbyl palmitate, and vitamin E acetate were added to the mixture. The mixture was stirred vigorously to allow it homogenized. Then, the mixture was allowed to cool to room temperature. The mixture was an opaque gel.

### EXAMPLE 20

This example is to formulate a nonaqueous ascorbic acid composition comprising methyl urea as the solubilization enhancer and vitamin E acetate as the oleaginous skin protectant in the propylene glycol. Methyl urea is available from Lancaster Synthesis (Ward Hill, Mass.). A nonaqueous composition in accordance with the invention was prepared as follows:

| Component | Amount (weight percentage) |
|---|---|
| Propylene glycol | 57% |
| Methyl urea | 15% |
| L-Ascorbic acid | 20% |
| Polysorbate-80 | 1.5% |
| Oleth-20 | 1.5% |
| Vitamin E acetate | 5% |

The propylene glycol was heated to 75.degree. C. The methyl urea and ascorbic acid were added together to the propylene glycol while maintaining the temperature at 75.degree. C., while stirring until dissolved. Polysorbate-80, oleth-20, and vitamin E acetate were added to the mixture. The mixture was stirred vigorously to allow it homogenized. Then, the mixture was allowed to cool to room temperature. The mixture was an opaque solution.

This example confirmed the solubilization enhancing effect of an alkyl urea.

### EXAMPLE 21

This example is to formulate a nonaqueous ascorbic acid composition comprising N-2-hydroxyethyl urea as the solubilization enhancer and vitamin E acetate as the oleaginous skin protectant in the propylene glycol. N-2-hydroxyethyl urea is available from a number of suppliers such as, for example, Aldrich Chemical Company (Milwaukee, Wis.). A nonaqueous composition in accordance with the invention was prepared as follows:

| Component | Amount (weight percentage) |
|---|---|
| Propylene glycol | 57% |
| N-2-hydroxyethyl urea | 15% |
| L-Ascorbic acid | 20% |
| Polysorbate-80 | 1.5% |
| Oleth-20 | 1.5% |
| Vitamin E acetate | 5% |

The propylene glycol was heated to 75.degree. C. The N-2-hydroxyethyl urea and ascorbic acid were added together to the propylene glycol while maintaining the temperature at 75.degree. C., while stirring until dissolved. Polysorbate-80, oleth-20 and vitamin E acetate were added to the mixture. The mixture was stirred vigorously to allow it homogenized. Then, the mixture was allowed to cool to room temperature. The mixture was an opaque solution.

This example confirmed the solubilization enhancing effect of a hydroxyalkyl urea.

### EXAMPLE 22

This example is to formulate a nonaqueous ascorbic acid composition comprising urea as the solubilization enhancer and vitamin E acetate as the oleaginous skin protectant in a mixture of propylene glycol and ethanol. This example is to demonstrate the preparation of the nonaqueous ascorbic acid composition in a mixture of a monohydric alcohol and polyol. In addition, ethanol is a known skin permeation enhancer, which could facilitate penetration of the active ingredients across skin barrier. A nonaqueous composition in accordance with the invention was prepared as follows:

| Component | Amount (weight percentage) |
|---|---|
| Propylene glycol | 52% |
| Ethanol | 5% |
| Urea | 15% |
| L-Ascorbic acid | 20% |
| Polysorbate-80 | 3% |
| Vitamin E acetate | 5% |

The mixture of propylene glycol and ethanol was heated to 75.degree. C in a reaction vessel equipped with a water-cooled condenser to minimize the loss of the volatile ethanol due to evaporation. The urea and ascorbic acid were added together to the mixture while maintaining the temperature at 75.degree. C., while stirring until dissolved. Polysorbate-80 and vitamin E acetate were added to the mixture. The mixture was stirred vigorously to allow it homogenized. Then, the mixture was allowed to cool to room temperature. The mixture was an opaque solution.

### EXAMPLE 23

This example is to formulate a nonaqueous ascorbic acid gel composition comprising urea as the solubilization enhancer and vitamin E acetate as the oleaginous skin protectant in propylene glycol. Carbopol Ultrez 20 was used as a thickening agent. Carbopol Ultrez 20 is available from Noveon (Cleveland, Ohio). A nonaqueous composition in accordance with the invention was prepared as follows:

| Component | Amount (weight percentage) |
|---|---|
| Propylene glycol | 55% |
| Urea | 15% |
| L-Ascorbic acid | 25% |
| Polysorbate-80 | 1.5% |
| Vitamin E acetate | 3% |
| Ultrez 20 | 0.5% |

Propylene glycol was heated to 75.degree. C. Urea and ascorbic acid were added together to the mixture while maintaining the temperature at 75.degree. C., while stirring until dissolved. Polysorbate-80 and vitamin E acetate were added to the mixture. The mixture was stirred vigorously to allow it homogenized. Then, Ultrez 20 was added in a shifted fashion. The mixture was stirred vigorously until Ultrez 20 was uniformly dispersed. Then, the mixture was allowed to cool to room temperature. The mixture was an opaque gel.

### EXAMPLE 24

This example is to formulate a nonaqueous ascorbic acid composition comprising urea as the solubilization enhancer and vitamin E acetate as the oleaginous skin protectant in propylene glycol. Vitamin A palmitate (Retinyl palmitate) was added as an anti-wrinkle agent. Retinyl palmitate is available from DSM Nutritional Products (Parsippany, N.J.). A nonaqueous composition in accordance with the invention was prepared as follows:

| Component | Amount (weight percentage) |
|---|---|
| Propylene glycol | 51.5% |
| Urea | 15% |
| L-Ascorbic acid | 25% |
| Polysorbate-80 | 2% |
| Oleth-20 | 1% |
| Vitamin E acetate | 5% |
| Vitamin A palmitate | 0.5% |

The propylene glycol was heated to 75.degree. C. The urea and ascorbic acid were added together to the mixture while maintaining the temperature at 75.degree. C., while stirring until dissolved. Polysorbate-80, oleth-20, and vitamin E acetate were added to the mixture. The mixture was stirred vigorously to allow it homogenized. The mixture was allowed to cool to about 50.degree. C. Vitamin A palmitate was added to the mixture at this temperature and dissolved. Then, the mixture was allowed to cool to room temperature. The mixture was an opaque solution.

### EXAMPLE 25

This example is to formulate a nonaqueous ascorbic acid composition comprising urea as the solubilization enhancer and vitamin E acetate as the oleaginous skin protectant in propylene glycol. A phospholipid (phosphatidyl choline, also known as lecithin), cholesterol, and a fatty acid (palmitic acid) were added to the composition. It is believed that phospholipids, cholesterol, and free fatty acids aid in restoration of damaged skin lipid barrier. A nonaqueous composition in accordance with the invention was prepared as follows:

| Component | Amount (weight percentage) |
|---|---|
| Propylene glycol | 53% |
| Urea | 12% |
| L-Ascorbic acid | 25% |
| Polysorbate-80 | 3% |
| Vitamin E acetate | 5% |
| Phosphatidyl choline | 0.5% |
| Cholesterol | 0.5% |
| Palmitic acid | 1.0% |

The propylene glycol was heated to 75.degree. C. The urea and ascorbic acid were added together to the mixture while maintaining the temperature at 75.degree. C., while stirring until dissolved. Polysorbate-80 and the phosphatidyl choline were added to the mixture at this temperature, while stirring until the phosphatidyl choline was solubilized. Then, vitamin E acetate, cholesterol, and palmitic acid were added to the mixture. The mixture was stirred to allow it homogenized. The mixture was allowed to cool to room temperature. The mixture was an opaque gel.

### EXAMPLE 26

This example is to formulate a nonaqueous ascorbic acid composition comprising urea as the solubilization enhancer and a mixture of lipid-soluble antioxidants as the oleaginous skin protectant in propylene glycol. A solution of coenzyme Q10 and vitamin E acetate dissolved in squalane was used to prepare the nonaqueous composition. This solution is available from Centerchem (Norwalk, Conn.) under name LIPOGARD. Coenzyme Q10 is a lipid-soluble ubiquinone antioxidant suitable for protecting against the damaging effect of free radicals, particularly the lipid peroxidation in skin. Squalane is moisturizing natural oil. A nonaqueous composition in accordance with the invention was prepared as follows:

| Component | Amount (weight percentage) |
|---|---|
| Propylene glycol | 60% |
| Urea | 12% |
| L-Ascorbic acid | 20% |
| Oleth-20 | 2% |
| Polysorbate-80 | 1% |
| LIPOGARD | 5% |

The propylene glycol was heated to 75.degree. C. The urea and ascorbic acid were added together to the mixture while maintaining the temperature at 75.degree. C., while stirring until dissolved. Oleth-20 and polysorbate-80 were then added to the mixture and solubilized. The mixture was allowed to cool to about 50.degree. C. LIPOGARD was added to the mixture at this temperature. The mixture was stirred vigorously to allow it homogenized. The mixture was allowed to cool to room temperature. The mixture was an opaque solution.

### EXAMPLE 27

The samples prepared in EXAMPLES 8, 11, 12, and 16 were selected as representatives for solubilization stability studies. The solubilization stability of a nonaqueous composition of the present invention is determined by observing if evidence of crystal formation or precipitation is evident at room temperature after a specified period of time. A nonaqueous composition is considered to be stable, in accordance with the present invention, when no evidence of crystal formation or precipitation is evident after one month. The solubilization enhancing effect in a nonaqueous composition is considered maintained when the following two criteria are met: (1). the nonaqueous composition is observed to be stable, and (2). the amount of ascorbic acid dissolved in the nonaqueous composition in the presence of the solubilization enhancer is greater than the amount of ascorbic acid that would be soluble in the nonaqueous alcohol solvent in the absence of the solubilization enhancer.

It was found that no evidence of crystal formation or precipitation was evident in the selected samples after one month. Thus, these samples are stable. In addition, the amount of ascorbic acid dissolved in the nonaqueous compositions prepared under various conditions, e.g., either at room temperature (EXAMPLE 8) or under heating condition (EXAMPLE 11, 12, or 16), is greater than the amount of ascorbic acid that would be soluble in the nonaqueous alcohol solvent in the absence of the solubilization enhancer under comparable conditions. The results can be seen in the comparison: EXAMPLE 5 vs. EXAMPLE 8, or EXAMPLE 1 vs. EXAMPLE 11, 12, or 16. Therefore, it is concluded that the solubilization enhancing effect is maintained in these nonaqueous compositions.

The studies confirmed that the oleaginous skin protectant at a concentration as high as 12% by weight has been incorporated into the nonaqueous compositions of the present invention, while maintaining the solubilization enhancing effect. Through judicious selection of the alcohol solvents, surfactants, solubilization enhancers, and combinations thereof, and solubilization conditions, concentrations higher than 12% by weight of the oleaginous skin protectants might also be incorporated into the nonaqueous compositions, while maintaining the solubilization enhancing effect.

It will thus be seen that the objects set forth above, among those made apparent from the preceding description, are efficiently attained and, since certain changes may be made in carrying out the above process and in the composition set forth without departing from the spirit and scope of the invention, it is intended that all matter contained in the above description shall be interpreted as illustrative and not in a limiting sense.

It is also to be understood that the following claims are intended to cover all of the generic and specific features of the invention herein described and all statements of the scope of the invention which, as a matter of language, might be said to fall there between.

Particularly it is to be understood that in the claims, ingredients or compounds recited in the singular are intended to include compatible combinations of such ingredients wherever the sense permits.

What is claimed is:

1. A method for preparing a nonaqueous ascorbic acid composition in an alcohol solvent having at least two carbon atoms, comprising:
placing ascorbic acid in intimate contact with the nonaqueous alcohol solvent containing a solubilization enhancer selected from the group consisting of urea, urea derivatives, and combinations thereof,
subjecting the nonaqueous alcohol solvent containing the solubilization enhancer and ascorbic acid to a temperature of from room temperature to about 160.degree. C. for sufficient time to permit the solubilization enhancer and ascorbic acid to dissolve in the nonaqueous alcohol solvent, yielding a nonaqueous mixture comprising the dissolved ascorbic acid and solubilization enhancer,
homogenizing the oleaginous skin protectant into the nonaqueous mixture to form a nonaqueous composition comprising the dissolved ascorbic acid, solubilization enhancer, and homogenized oleaginous skin protectant,
wherein the amount of ascorbic acid dissolved in the nonaqueous mixture in the presence of the solubilization enhancer is greater than the amount of ascorbic acid that would be soluble in the nonaqueous alcohol solvent in the absence of the solubilization, and the solubilization enhancing effect is maintained after the oleaginous skin protectant is homogenized into the nonaqueous mixture.

2. The method of claim 1 wherein the solubilization enhancer comprises urea.

3. The method of claim 1 wherein the urea derivatives are selected from the group consisting of mono-substituted alkyl, hydroxyalkyl ureas, and combinations thereof.

4. The method of claim 1 wherein the nonaqueous alcohol solvent, ascorbic acid, and solubilization enhancer are heated to a temperature of from about 50.degree. C. to about 120.degree. C. for sufficient time to permit the solubilization enhancer and ascorbic acid to dissolve in the nonaqueous alcohol solvent.

5. The method of claim 1 wherein the nonaqueous alcohol solvent comprises polyol.

6. The method of claim 1 wherein the ingredients of the nonaqueous composition and solubilization conditions are selected so that the solubilization enhancing effect is maintained after the oleaginous skin protectant is homogenized into the nonaqueous mixture.

7. A method for preparing a nonaqueous ascorbic acid composition in an alcohol solvent having at least two carbon atoms, comprising:
providing in intimate contact, a quantity of the nonaqueous alcohol solvent and an effective amount of a solubilization enhancer selected from the group consisting of urea, urea derivatives, and combinations thereof,
mixing the solubilization enhancer and nonaqueous alcohol solvent at a temperature of from room temperature to about 160.degree. C. for sufficient time to permit the solubilization enhancer to dissolve in the nonaqueous alcohol solvent to form a solubilization enhancer solution,
combining the solubilization enhancer solution and a quantity of ascorbic acid,
subjecting the combination to a temperature of from room temperature to about 160.degree. C. for sufficient time to permit the ascorbic acid to dissolve in the solubilization enhancer solution, yielding a nonaqueous mixture comprising the dissolved ascorbic acid and solubilization enhancer,
homogenizing the oleaginous skin protectant into the nonaqueous mixture to form a nonaqueous composition comprising the dissolved ascorbic acid, solubilization enhancer, and homogenized oleaginous skin protectant,
wherein the amount of ascorbic acid dissolved in the nonaqueous mixture in the presence of the solubilization enhancer is greater than the amount of ascorbic acid that would be soluble in the nonaqueous alcohol solvent in the absence of the solubilization, and the solubilization enhancing effect is maintained after the oleaginous skin protectant is homogenized into the nonaqueous mixture.

8. The method of claim 7 wherein the solubilization enhancer comprises urea.

9. The method of claim 7 wherein the solubilization enhancer comprises mono-substituted ureas.

10. The method of claim 7 wherein the nonaqueous alcohol solvent comprises combinations of a monohydric alcohol and polyol.

11. The method of claim 7 wherein the nonaqueous alcohol solvent comprises polyol.

12. The method of claim 7 wherein the solubilization process is conducted at a temperature of from about 50.degree. C. to about 120.degree. C.

13. The method of claim 7 wherein the oleaginous skin protectant is selected from the group consisting of antioxidant, moisturizing compound, dermatologically active compound, sunscreen, aesthetic agent, and combinations thereof.

14. A nonaqueous composition, comprising, by weight of the total composition:
ascorbic acid, in an amount of about 5 to about 40%,
a solubilization enhancer selected from the group consisting of urea, urea derivatives, and combinations thereof, in an amount of about 1 to about 40%,
an oleaginous skin protectant, in amount of about 0.01 to about 40%,
a nonaqueous alcohol solvent, in an amount of about 20 to about 90%,
wherein the oleaginous skin protectant is homogenized in the nonaqueous composition comprising the dissolved ascorbic acid, solubilization enhancer, and nonaqueous alcohol solvent, and the amount of ascorbic acid dissolved in the nonaqueous composition in the presence of the solubilization enhancer is greater than the amount of ascorbic acid that would be soluble in the nonaqueous alcohol solvent in the absence of the solubilization enhancer, and the solubilization enhancing effect is maintained.

15. The composition of claim 14 wherein the nonaqueous alcohol solvent comprises polyol.

16. The composition of claim 15 wherein the polyol is propylene glycol.

17. The composition of claim 14 wherein the solubilization enhancer is urea.

18. The composition of claim 14 wherein the urea derivatives are selected from the group consisting of mono-substituted alkyl, hydroxyalkyl ureas, and combinations thereof.

19. The composition of claim 14 wherein the oleaginous skin protectant is selected from the group consisting of antioxidant, moisturizing compound, dermatologically active compound, sunscreen, aesthetic agent, and combinations thereof.

20. The composition of claim 19 wherein the antioxidant is selected from the group consisting of vitamin E and derivatives thereof, vitamin A and derivatives thereof, coenzyme Q10, lipoic acid, ascorbic acid alkanoates, and combinations thereof.
